(19) United States
(12) Patent Application Publication
(10) Pub. No.: US 2022/0349885 A1
(43) Pub. Date: Nov. 3, 2022

(54) Biomarker for diagnosing rheumatoid arthritis and uses thereof

(71) Applicant: EULJI UNIVERSITY INDUSTRY ACADEMIC COOPERATION FOUNDATION, Gyeonggi-do (KR)

(72) Inventors: Hee-Gyoo KANG, Seoul (KR); Jiyeong LEE, Gyeonggi-do (KR); Sora MUN, Seoul (KR); Mi-Kyoung LIM, Daejeon (KR)

(21) Appl. No.: 17/387,396

(22) Filed: Jul. 28, 2021

(30) Foreign Application Priority Data
Apr. 30, 2021 (KR) 10-2021-0056700

(51) Int. Cl. G01N 33/564; G01N 33/68

(52) U.S. Cl. CPC G01N 33/564; G01N 33/6893; G01N 2333/4706; G01N 2333/914; G01N 2800/102; G01N 2800/24

(57) ABSTRACT

The present invention relates to a marker composition for diagnosing rheumatoid arthritis, comprising angiotensinogen (ACT) as a marker, a method for providing information necessary to determine the occurrence of rheumatoid arthritis using the marker composition, a composition for determining the occurrence of rheumatoid arthritis, comprising an agent for measurement of the expression level of the marker, and a kit for determining the occurrence of rheumatoid arthritis, comprising a device for measurement of the expression level of the marker. The method for providing information for use in determining the occurrence of rheumatoid arthritis provided by the present invention can be widely utilized to determine the occurrence of various joint diseases, including rheumatoid arthritis since it is possible to measure the expression levels of proteins of which the expression levels are changed at the time of the occurrence of rheumatoid arthritis, and to more objectively and accurately determine the occurrence of rheumatoid arthritis when the method is used.

### TECHNICAL FIELD

The present invention relates to a biomarker for diagnosing rheumatoid arthritis and uses thereof. More specifically, the present invention relates to a marker composition for diagnosing rheumatoid arthritis, comprising angiotensinogen (ACT) as a marker, a method for providing information necessary to determine the occurrence of rheumatoid arthritis using the marker composition, a composition for determining the occurrence of rheumatoid arthritis, comprising an agent for measurement of the expression level of the marker, and a kit for determining the occurrence of rheumatoid arthritis, comprising a device for measurement of the expression level of the marker.

### BACKGROUND ART

Rheumatoid arthritis is a kind of autoimmune disease caused by various causes such as genetic predisposition, smoking, and infectious diseases such as periodontitis, and is a chronic inflammatory disease in which autoantibodies are generated due to such causes and lymphocytes continuously attack the joint synovial membrane via the generated autoantibodies. In terms of age, rheumatoid arthritis mainly occurs between the ages of 35 and 50, the incidence rate in women is three times higher than in men, and the disease is known to be accompanied by various symptoms such as pain, fatigue, and rheumatoid arthritis. In addition, it is known that rheumatoid arthritis may be classified according to the detection of RF (rheumatoid factor) and anti-CPP (cyclic citrullinated peptide) antibody present in the blood of patients.

As a method for determining the occurrence of rheumatoid arthritis, a method has been used in which the presence or absence of RF and anti-CPP antibody present in the blood of patients is detected. However, it is known that the level of RF in the blood increases at the time of the occurrence of various joint diseases or autoimmune diseases other than rheumatoid arthritis as well, and the RF has a disadvantage in that the false positive rate is high, and the anti-CPP antibody exhibits significantly low sensitivity, and can thus be used as an indicator that does not mistake a normal person for a patient, but has a disadvantage in that the anti-CPP antibody is inappropriate for use as an indicator for diagnosing patients with rheumatoid arthritis.

Accordingly, research to develop a method for diagnosing rheumatoid arthritis more accurately and effectively has been actively conducted. For example, Korean Patent No. 10-1978677 discloses a method for diagnosing rheumatoid arthritis using IL-6, CCL-2, and IL-8 as markers, and Japanese Patent No. 6663000 discloses a method for diagnosing rheumatoid arthritis using immunoreactivity to filamin A and N-acetylglucosamine-6-sulfatase. However, since these methods depend on the level of the patient's immune response, there is a problem in that it is difficult to normally perform diagnosis when the immune response is activated by another disease.

With this background, the present inventors have made intensive research efforts to develop a method for diagnosing rheumatoid arthritis more objectively without depending on the patient's immune response, and as a result, confirmed that the occurrence of rheumatoid arthritis can be more objectively determined by the expression level of angiotensinogen (ACT) contained in serum, thereby completing the present invention.

### DISCLOSURE

### Technical Problem

A main object of the present invention is to provide a marker composition for diagnosing rheumatoid arthritis, comprising angiotensinogen as a marker.

Another object of the present invention is to provide a method for providing information necessary to determine the occurrence of rheumatoid arthritis using the marker composition.

Still another object of the present invention is to provide a composition for determining the occurrence of rheumatoid arthritis, comprising an agent for measurement of the expression level of the marker.

Still another object of the present invention is to provide a kit for determining the occurrence of rheumatoid arthritis, comprising a device for measurement of the expression level of the marker.

Still another object of the present invention is to provide the use of angiotensinogen to diagnose rheumatoid arthritis.

### Technical Solution

Each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description below.

Further, those skilled in the art will recognize, or be able to ascertain using no more than routine experimentation, many equivalents to the specific embodiments of the invention described herein. Further, these equivalents should be interpreted to fall within the scope of the present invention.

In addition, throughout this specification, when a part is referred to as “including” an element, it will be understood that other elements may be further included rather than other elements being excluded unless content to the contrary is specially described.

Hereinafter, the present invention will be described in detail.

An embodiment of the present invention for achieving the objects provides a marker composition for diagnosing rheumatoid arthritis, comprising angiotensinogen (AGT) as a marker.

In order to develop a method for objectively evaluating and determining the occurrence of rheumatoid arthritis, the present inventors have conducted various studies to discover protein markers of which the expression levels change depending on the occurrence of rheumatoid arthritis among proteins contained in the serum. As a result, the present inventors have confirmed that the expression level of angiotensinogen changes depending on the occurrence of rheumatoid arthritis, and discovered angiotensinogen as a marker.

As used herein, the term “rheumatoid arthritis” refers to a kind of autoimmune disease caused by various causes, such as genetic predisposition, smoking, and infectious diseases such as periodontitis, and refers to a chronic inflammatory disease in which autoantibodies are generated by the causes and lymphocytes continuously attack the joint synovial membrane by the generated autoantibodies.

As used herein, the term “diagnosis” includes the act of determining the susceptibility of a subject to a particular disease or illness; the act of determining whether a subject currently has a particular disease or illness; the act of determining the prognosis of a subject having a particular disease or illness; or therametrics (for example, the act of monitoring the condition of a subject to provide information on treatment efficacy).

In the present invention, the diagnosis of rheumatoid arthritis may be interpreted as meaning an act of objectively determining whether rheumatoid arthritis has occurred in a targeted patient.

As used herein, the term “angiotensinogen (AGT)” refers to a precursor of angiotensin that serves to increase blood pressure in the body. The specific amino add sequence of the angiotensinogen or the nucleotide sequence information of a gene encoding the same is reported in a database such as the NCBI. For example, the sequence is reported as GenBank Accession Nos. NP_001171966.1, AH003514.2. and the like.

Meanwhile, as the marker protein for diagnosing rheumatoid arthritis, retinol-binding protein 4 (RBP4), serum amyloid A-4 (SAA4), or vitamin D-binding protein (VDBP) may be additionally used in addition to the angiotensinogen.

As used herein, the term “retinol-binding protein 4 (RBP4)” refers to a protein belonging to the group of binding proteins that bind to retinol, and refers to a protein responsible for the transport and metabolic regulation of retinol, which is known to regulate overall development and gene expression of the embryo during early embryonic development. The specific amino add sequence of the retinol-binding protein 4 or the nucleotide sequence information of a gene encoding the same is reported in a database such as the NCBI. For example, the sequence is reported as GenBank Accession Nos. AKI71630.1, NM_001311257, KR711248.1, and the like.

As used herein, the term “serum amyloid A-4 (SAA4)” refers to a kind of acute inflammatory protein, and is known to increase rapidly in immune diseases such as rheumatoid arthritis. The specific amino add sequence of the serum amyloid A-4 or the nucleotide sequence information of a gene encoding the same is reported in a database such as the NCBI. For example, the sequence is reported as GenBank Accession Nos. CAG46558.1, CR541758.1, and the like.

As used herein, the term “vitamin D-binding protein (VDBP)” refers to a protein that binds to vitamin D, and is known to play a role of actin scavenging protein to remove F-actin, and play a role of protecting the mechanism of damage and destruction of joint tissue through an anti-inflammatory reaction in tissues. The specific amino acid sequence of the vitamin D-binding protein or the nucleotide sequence information of a gene encoding the same is reported in a database such as the NCBI. For example, the sequence is reported as Gen Bank Accession Nos. NP_036696.2, NM_012564.3, and the like.

Another embodiment of the present invention provides a method for providing information necessary to determine the occurrence of rheumatoid arthritis using the marker composition.

Specifically, the method for providing information necessary to determine the occurrence of rheumatoid arthritis according to the present invention includes quantitatively analyzing the expression level of a marker protein selected from the group consisting of angiotensinogen, retinol-binding protein 4, and a combination thereof in a serum sample of an individual suspected of having rheumatoid arthritis.

As used herein, the term “individual” may include, without limitation, mammals including rats, livestock, and humans, farmed fish, and the like, which are likely to develop or have rheumatoid arthritis.

As described above, in addition to the angiotensinogen, retinol-binding protein 4, serum amyloid A-4, and vitamin D-binding protein may be additionally used.

In the present invention, any method known to those skilled in the art may be used for the step of quantitatively analyzing the expression level of a protein. As a specific example, PCR, ligase chain reaction (LCR), transcription amplification, self-sustained sequence replication, nucleic add sequence-based amplification (NASBA) method, and the like may be used, but the analysis method is not limited thereto. Here, the amino add sequence of the marker protein for diagnosis of rheumatoid arthritis according to the present invention or the nucleotide sequence of a gene encoding the same is known in a database such as the NCBI, and those skilled in the art may use appropriate means required to measure the protein expression level.

As described above, the method may further comprise quantitatively analyzing the expression levels of retinol-binding protein 4, serum amyloid A-4, and vitamin D-binding protein in addition to the angiotensinogen.

Meanwhile, the method may further comprise correlating the levels of angiotensinogen, retinol-binding protein 4, serum amyloid A-4, and vitamin D-binding protein, which are proteins quantitatively analyzed from the serum sample, with the determination of the occurrence of rheumatoid arthritis.

In other words, since there is a deviation in the quantitatively analyzed level of each protein depending on the condition of the patient, it is not easy to use only the quantitatively analyzed fragmentary level of a protein to determine the occurrence of rheumatoid arthritis, and thus the quantitatively analyzed levels of the respective proteins may be analyzed in combination to determine the occurrence of rheumatoid arthritis.

As an example of the method for analyzing the quantitative analysis results of the respective proteins in combination, it is possible to use a method for determining the occurrence of rheumatoid arthritis by using the quantitatively analyzed levels of angiotensinogen, retinol-binding protein 4, serum amyloid A-4, and vitamin D-binding protein measured from a serum sample singly or in combination.

As another example of the method for analyzing the quantitative analysis results of the respective proteins in combination, conventional statistical analysis methods may be used. Here, the statistical analysis method that can be used is not particularly limited as long as it can determine the blood-stained time. As an example, a logistic regression analysis method; a linear or nonlinear regression analysis method; a linear or nonlinear classification analysis method; ANOVA; a neural network analysis method; a genetic analysis method; a support vector machine analysis method; a hierarchical cluster analysis or cluster analysis method; a hierarchical algorithm using decision trees, or Kernel principal component analysis method; a Markov Blanket analysis method; a recursive feature elimination or entropy-based recursive feature elimination analysis method; a forward floating search or backward floating search analysis method; and the like may be used singly or in combination.

The combination of the quantitative analysis results may be performed using a computer algorithm capable of automatically performing the statistical methods.

Still another embodiment of the present invention provides a composition for determining the occurrence of rheumatoid arthritis, comprising an agent for measurement of the expression level of angiotensinogen.

As used herein, the term “agent for measurement of the quantitatively analyzed level” refers to an agent capable of specifically binding to and recognizing the protein or mRNA encoding the same or amplifying the miRNA. As a specific example, the agent may be an antibody that specifically binds to the protein, or a primer or probe that specifically binds to the miRNA, but is not limited thereto, and those skilled in the art will be able to select an appropriate agent for the purpose of the invention.

The agent may be labeled directly or indirectly to measure the expression level of the protein or mRNA. Specifically, ligands, beads, radionuclides, enzymes, substrates, cofactors, inhibitors, fluorescers, chemiluminescent materials, magnetic particles, haptens and dyes may be used as the label, but the label is not limited thereto. As specific examples, the ligands include biotin, avidin, and streptavidin, the enzymes include luciferase, peroxidase, and beta galactosidase, and the fluorescers include fluorescein, coumarin, rhodamine, phycoerythrin, and sulforhodamine 101 add chloride (Texas Red), but the label is not limited thereto. Most known labels may be used as such detectable labels, and those skilled in the art will be able to select an appropriate label for the purpose of the invention.

As used herein, the term “antibody” refers to a proteinaceous molecule capable of specifically binding to an antigenic site of a protein or peptide molecule. Such an antibody can be prepared by a conventional method from a protein to be encoded by the marker gene, the protein being obtained by cloning each gene into an expression vector according to a conventional method. The form of the antibody is not particularly limited, and a part thereof is also included in the antibody of the present invention as long as it has a polyclonal antibody, a monoclonal antibody, or antigen-binding property. Not only all immunoglobulin antibodies but also special antibodies such as humanized antibodies may be included. In addition, the antibody includes functional fragments of antibody molecules as well as complete forms having two full-length light chains and two full-length heavy chains. A functional fragment of an antibody molecule means a fragment having at least an antigen-binding function, and may be Fab, F(ab′), F(ab′)₂, and Fv.

As used herein, the term “primer” refers to a nucleotide sequence having a short free 3′ hydroxyl group, and refers to a short sequence capable of forming a base pair with a complementary template and functioning as a starting point for template strand copying. in the present invention, the primer used for the miRNA amplification may be a single-stranded oligonucleotide that can act as a starting point for template-directed DNA synthesis in an appropriate buffer at an appropriate temperature under appropriate conditions (for example, four different nucleoside triphosphates and a polymerase such as DNA/RNA polymerase or reverse transcriptase). The appropriate length of the primer may vary depending on the purpose of use. The primer sequence is not required to be completely complementary to the polynucleotide of the miRNA of the gene or its complementary polynucleotide, and any primer can be used as long as the primer sequence is sufficiently complementary to hybridize.

As used herein, the term “probe” refers to a labeled nucleic acid fragment or peptide capable of specifically binding to miRNA. As specific examples, the probe may be constructed in the form of an oligonucleotide probe, a single stranded DNA probe, a double stranded DNA probe, an RNA probe, an oligonucleotide peptide probe, a polypeptide probe, or the like.

The composition may further comprise agents for measurement of the expression levels of retinol-binding protein 4, serum amyloid A-4, and vitamin D-binding protein in addition to the agent for measurement of the expression level of angiotensinogen as described above.

Still another embodiment of the present invention provides a kit for determining the occurrence of rheumatoid arthritis, comprising a quantitative device for measurement of the expression level of angiotensinogen.

The quantitative device included in the diagnostic kit of the present invention may measure the expression level of the marker protein. As a specific example, the quantitative device may be an RT-PCR kit or an ELISA kit, but is not limited thereto as long as the expression level of miRNA or protein can be measured.

In this case, the RT-PCR kit may be a kit including essential elements necessary to perform RT-PCR. For example, the RT-PCR kit may contain, in addition to each primer specific for the gene, a test tube or another suitable container, reaction buffers (pH and magnesium concentrations vary), deoxynucieotides (dNTPs), dideoxynucleotides (ddNTPs), enzymes such as Tag-polymerase and reverse transcriptase, DNase, RNAse inhibitors, DEPC-water, sterile water, and the like. In addition, a primer pair specific for the gene used as a quantitative control may be contained.

The kit may further comprise quantitative devices for measurement of the expression levels of retinol-binding protein 4, serum amyloid A-4, and vitamin D-binding protein in addition to the quantitative device for measurement of the expression level of angiotensinogen as described above.

Still another embodiment of the present invention provides the use of angiotensinogen to determine the occurrence of rheumatoid arthritis.

### Advantageous Effects

The method for providing information for use in determining the occurrence of rheumatoid arthritis provided by the present invention can be widely utilized to determine the occurrence of various joint diseases, including rheumatoid arthritis since it is possible to measure the expression levels of proteins of which the expression levels are changed at the time of the occurrence of rheumatoid arthritis, and to more objectively and accurately determine the occurrence of rheumatoid arthritis when the method is used.

### BRIEF DESCRIPTION OF DRAWINGS

FIG. 1A is a graph illustrating the results of principal component analysis performed on unique proteins contained in the samples of a control group and an experimental group;

FIG. 1B is a graph illustrating proteins having an expression level changed by 2-fold or more and a p-value of 0.05 or less at the same time when volcano plot analysis is performed on proteins contained in the samples of a control group and an experimental group;

FIG. 1C is a diagram illustrating the results of heatmap analysis performed on proteins having an expression level changed by 2-fold or more, which are proteins contained in the samples of a control group and an experimental group;

FIGS. 2A-G are extracted ion chromatograms (EIC) of peptides used for absolute MRM quantification of angiotensinogen (A), C-reactive protein (B), gelsolin (C), lymphatic vessel endothelial hyaluronan receptor 1 (D), retinol-binding protein 4 (E), serum amyloid A-4 (F), and VDBP (G) contained in the serum samples of an experimental group;

FIG. 3A is a graph illustrating the concentration of angiotensinogen contained in the serum samples of a control group and an experimental group;

FIG. 3B is a graph illustrating the concentration of serum amyloid A-4 contained in the serum samples of a control group and an experimental group;

FIG. 3C is a graph illustrating the concentration of retinol-binding protein 4 contained in the serum samples of a control group and an experimental group;

FIG. 3D is a graph illustrating the concentration of vitamin D-binding protein contained in the serum samples of a control group and an experimental group;

FIG. 4A is a graph illustrating the concentration of angiotensinogen contained in the serum samples of a control group and each type of patients with rheumatoid arthritis;

FIG. 4B is a graph illustrating the concentration of serum amyloid A-4 contained in the serum samples of a control group and each type of patients with rheumatoid arthritis;

FIG. 4C is a graph illustrating the concentration of retinol-binding protein 4 contained in the serum samples of a control group and each type of patients with rheumatoid arthritis;

FIG. 4D is a graph illustrating the concentration of vitamin D-binding protein contained in the serum samples of a control group and each type of patients with rheumatoid arthritis;

FIG. 5A is an ROC graph illustrating the results of logistic regression analysis performed on the expression levels of four biomarkers (angiotensinogen, serum amyloid A-4, retinol-binding protein 4, and vitamin D-binding protein) in the serum samples of a control group and an experimental group; and

FIG. 5B is an ROC graph illustrating the results of logistic regression analysis performed on a combination of the expression levels of four biomarkers (angiotensinogen, serum amyloid A-4, retinol-binding protein 4, and vitamin D-binding protein) in the serum samples of a control group and an experimental group.

### DETAILED DESCRIPTION OF THE INVENTION

Hereinafter, the configuration and effects of the present invention will be described in more detail with reference to exemplary embodiments. However, these exemplary embodiments are for illustrative purposes only, and the scope of the present invention is not intended to be limited by these exemplary embodiments.

### Example 1: Preparation of Samples

Serum samples of 251 patients with rheumatoid arthritis (experimental group) and serum samples of 230 healthy controls (control group) were collected from the Eulji University Hospital Institutional Review Board.

Roughly, each blood sample taken from the patients with rheumatoid arthritis and the healthy controls was left at 24° C. for 2 hours, and then centrifuged (4,000×g, 5 min) to obtain each serum.

The obtained serum was applied to an LC column (human 6-HC, 4.6 mm×50 mm; Agilent Technologies, Santa Clara, Calif., USA) to deplete serum proteins (albumin, IgG, antitrypsin, IgA, transferrin, and haptoglobin) known to be highly abundantly contained in the blood, and then applied to a Nanosep device equipped with a polyether sulfone membrane 3K (Pall, Zaventem, Belgium) to be concentrated. The concentrated sample was applied to a mass spectrometer (AB Sciex 5600, Framingham, Mass., USA) and MRM (multiple reaction monitoring)-based targeted protein quantification to select candidate biomarkers for diagnosis of rheumatoid arthritis.

At this time, the MRM-based targeted protein quantification result was evaluated by Welch's correction T test method using GraphPad Prism version 8.0 for Windows (GraphPad Software Inc., San Diego, Calif.), and logistic regression analysis method using SPSS software package version 18.0.0 (SPSS Inc., Chicago, Ill., USA) was employed,

### Example 2: Processing of Samples

To the serum sample (100 μg of serum protein) obtained in Example 1, 5 mM tris(2-carboxyethyl)phosphine (Pierce Chemical Company, Rockford, Ill., USA) was added, the reaction was conducted for 30 minutes at 37° C. and 300 rpm, 15 mM iodoacetamide (Sigma-Aldrich, St. Louis, Mo., USA) was further added, the reaction was conducted again for 1 hour at 24° C. and 300 rpm under dark conditions for alkylation.

Subsequently, the alkylated sample was treated with mass spectrometry-grade trypsin gold (Promega Corporation, Fitchburg, Wis., USA), and reacted at 37° C. overnight to cleave the serum proteins into peptides.

The cleaved peptide sample was applied to the OFFGEL fractionator (3100 OFFGEL Low Res Kit, pH 3-10; Agilent Technologies, Santa Clara, Calif., USA), and separated into 12 fractions through pH 3-10 isoelectric points.

A sample of each of the separated fractions was loaded onto the Eksigent nanoLC 400 system and cHiPLC (AB Sciex, Concord, ON, Canada) for analysis, and analyzed using a TripleTOF 5600 mass spectrometer (AB Sciex).

At this time, the sample (1 μg/μL) was injected into the Eksigent ChromXP nanoLC trap column (350 μm i.d.×0.5 mm, ChromXP C 18 3 μm) at a flow rate of 5000 nL/min, and eluted from the Eksigent ChromXP nanoLC column (75 μm i.d.×15 cm) at a flow rate of 300 nL/min for 120 min, and then mobile phase B buffer was added at a gradient of 5% to 90% for 120 min (0 min/5%, 10.5 min/40%, 105.5 min/90%, 111.5 min/90%, 112 min/5%, and 120 min5%).

As a result, a total of 339 proteins were identified in the samples of the control group and the experimental group, 194 unique proteins were identified in the control samples, and 111 unique proteins were identified in the experimental group samples.

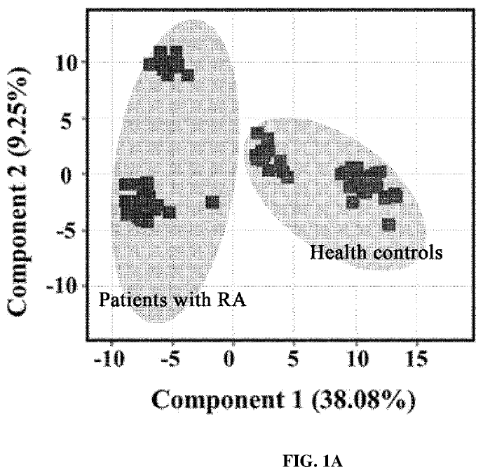

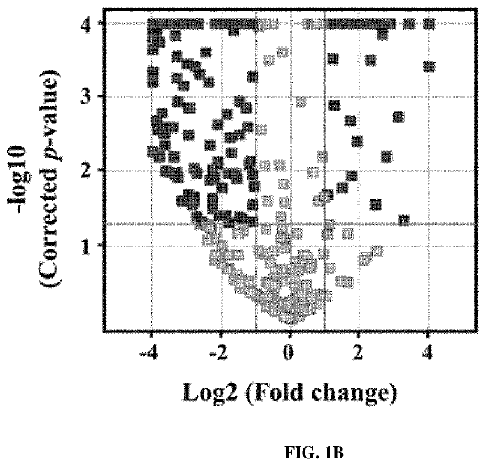

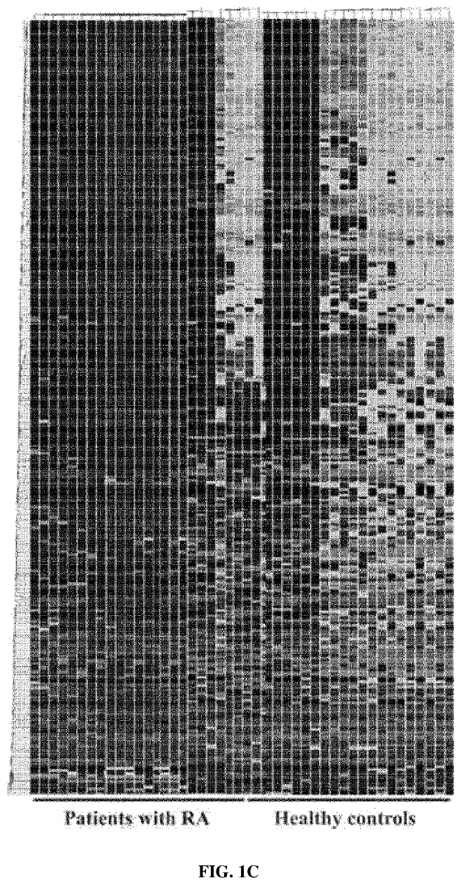

Thereafter, SWATH analysis was performed to quantify the identified proteins, and then principal component analysis (PCA) was performed using the quantification data acquired from the analysis (FIG. 1A).

FIG. 1A is a graph illustrating the results of principal component analysis performed on the unique proteins contained in the samples of the control group and the experimental group.

As illustrated in FIG. 1A, it was confirmed that the unique proteins contained in the samples of the control group and the experimental group were clearly distinguished from each other.

PLS-DA (partial least squares discriminant analysis) was performed using the analysis results of the samples of the control group and the experimental group, filtering was performed by the p value (p<0.05), and duster analysis was performed on the proteins having an expression level changed by 2-fold or more (FIG. 1B).

FIG. 1B is a graph illustrating proteins having an expression level changed by 2-fold or more and a p-value of 0.05 or less at the same time when volcano plot analysis is performed on the proteins contained in the samples of the control group and the experimental group.

As illustrated in FIG. 1B, it was confirmed that the samples of the control group and the experimental group contained a large number of proteins of which the expression levels were upregulated or downregulated by 2-fold or more.

Heatmap analysis was performed on the proteins having an expression level changed by 2-fold or more, which were obtained as a result of the duster analysis (FIG. 1C).

FIG. 1C is a diagram illustrating the results of heatmap analysis performed on the proteins having an expression level changed by 2-fold or more, which were contained in the samples of the control group and the experimental group.

As illustrated in FIG. 1C, it was confirmed that a larger number of proteins having a significantly upregulated expression level was present in the samples of the experimental group than in the samples of the control group.

### Example 3: Selection of Biomarkers

From the results of heatmap analysis performed in Example 2, it was attempted to select biomarkers of which the expression levels were upregulated in the samples of the experimental group than in the samples of the control group.

First, seven proteins having a significantly upregulated expression level in the samples of the experimental group compared to in the samples of the control group were first selected as biomarker candidates. Here, the selected seven proteins are angiotensinogen (AGT), C-reactive protein, gelsolin, lymphatic vessel endothelial hyaluronan receptor 1, retinal-binding protein 4 (RBP4), serum amyloid A-4 (SAA4), and vitamin D-binding protein (VDBP).

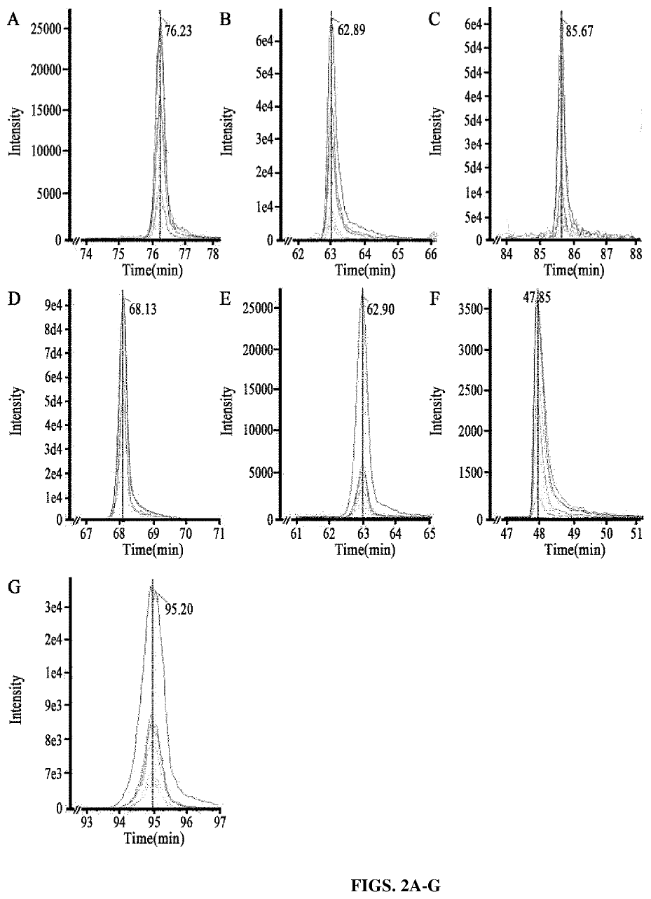

Extracted ion chromatography of selected peptides was performed to absolutely quantify the seven selected proteins, which were contained in the serum samples of the experimental group (FIG. 2).

FIG. 2 is extracted ion chromatograms (EIC) of peptides used for absolute MRM quantification of angiotensinogen (A), C-reactive protein (B), gelsolin (C), lymphatic vessel endothelial hyaluronan receptor 1 (D), retinol-binding protein 4 (E), serum amyloid A-4 (F), and VDBP (G) contained in the serum samples of the experimental group.

The area under the quantitative curve (AUC) of each protein acquired in FIG. 2 was measured, and it was confirmed that (A) angiotensinogen (AUC=0.8346), (B) C-reactive protein (AUC=0.5030), (C) gelsolin (AUC=0.6794), (D) lymphatic vessel endothelial hyaluronan receptor 1 (AUC=0.5309), (E) retinol-binding protein 4 (AUC=0.9391), (F) serum amyloid A-4 (AUC)=0.8994), and (G) vitamin D-binding protein (AUC=0.8170), respectively.

From the results, four proteins having an AUC value of 0.8 or more, including (A) angiotensinogen (AUC=0.8346), (E) retinol-binding protein 4 (AUC=0.9391), (F) serum amyloid A-4 (AUC)=0.8994), and (G) vitamin D-binding protein (AUC=0.8170) were ultimately selected as biomarkers.

### Example 4: Validation of Effect of Biomarkers for Diagnosis of Rheumatoid Arthritis

### Example 4-1: Comparison Between Healthy Control and Patient with Rheumatoid Arthritis

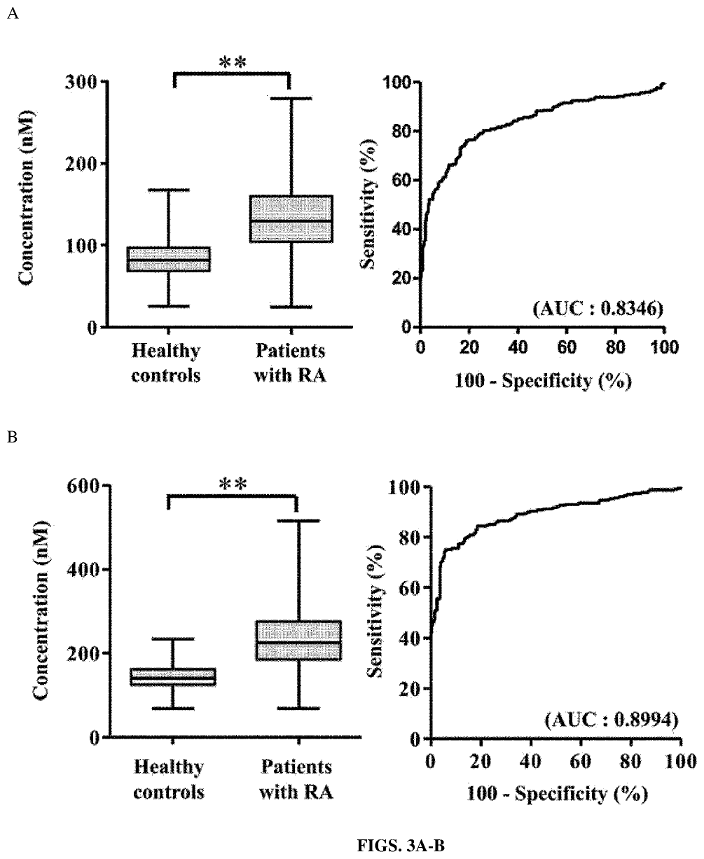

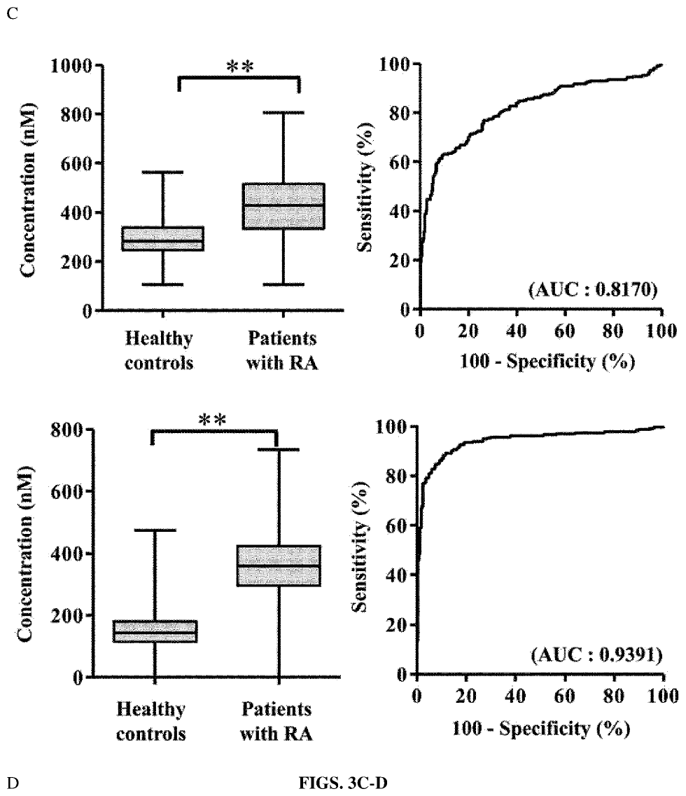

The expression levels of the four biomarkers selected in Example 3 in the serum samples of the control group and the experimental group collected in Example 1 were measured and compared (FIGS. 3A to 3D). Here, the comparison results of the expression levels of the respective biomarkers were displayed as box-and-whisker plots.

FIG. 3A illustrates the concentration of angiotensinogen contained in the serum samples of the control group and the experimental group, FIG. 3B illustrates the concentration of serum amyloid A-4 contained in the serum samples of the control group and the experimental group, FIG. 3C illustrates the concentration of retinol-binding protein 4 contained in the serum samples of the control group and the experimental group, and FIG. 3D illustrates the concentration of vitamin D-binding protein contained in the serum samples of the control group and the experimental group.

As illustrated in FIGS. 3A to 3D, it was confirmed that all of the four biomarkers selected in Example 3 were detected at higher levels in the sera of the experimental group than in the sera of the control group.

### Example 4-2: Comparison According to Type of Patient with Rheumatoid Arthritis

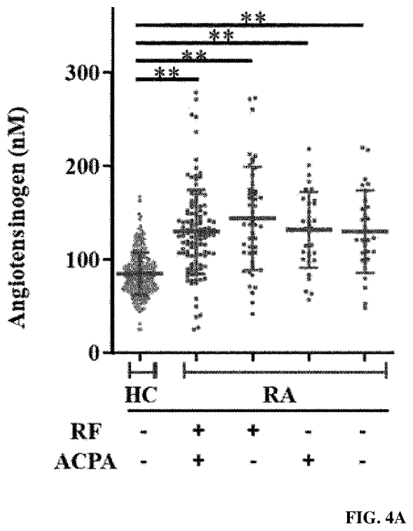

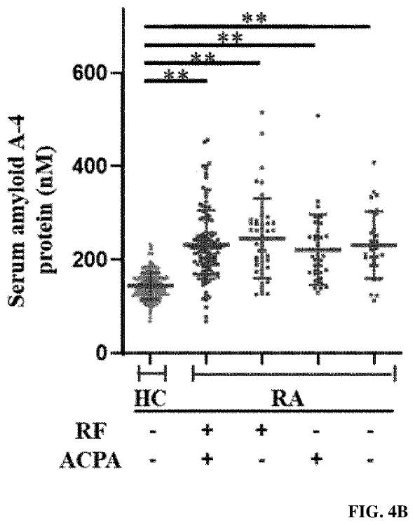

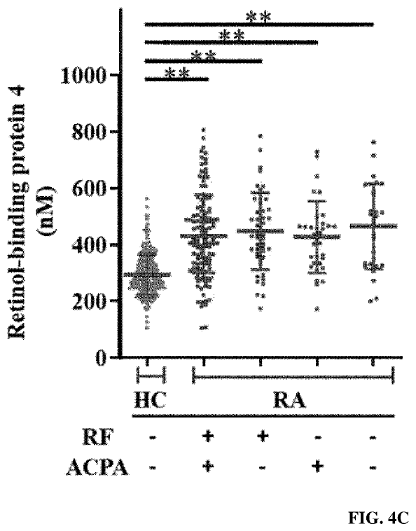

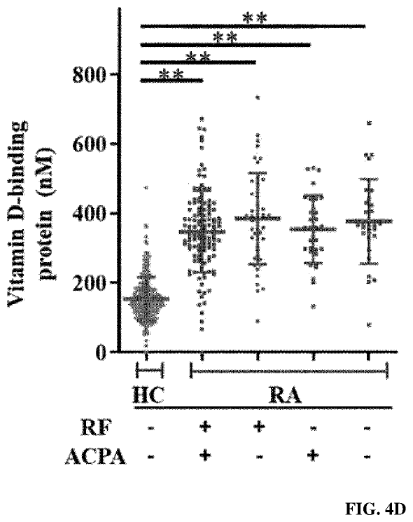

In general, it is known that rheumatoid arthritis is divided into a type which is negative for each of the RF (rheumatoid factor) and anti-CPP (cyclic citrullinated peptide) antibody, and a type which is positive for each of the RF and anti-CPP antibody, and the expression levels of the four biomarkers selected in Example 3 in the serum samples of each type of patients with rheumatoid arthritis were measured and compared (FIGS. 4A to 4D). Here, serum samples of healthy controls (HC) were used as the control group, and the comparison results of the expression levels of the respective biomarkers were displayed as scatter plots.

FIG. 4A illustrates the concentration of angiotensinogen contained in the serum samples of the control group and each type of patients with rheumatoid arthritis, FIG. 4B illustrates the concentration of serum amyloid A-4 contained in the serum samples of the control group and each type of patients with rheumatoid arthritis, FIG. 40 illustrates the concentration of retinol-binding protein 4 contained in the serum samples of the control group and each type of patients with rheumatoid arthritis, and FIG. 40 illustrates the concentration of vitamin D-binding protein contained in the serum samples of the control group and each type of patients with rheumatoid arthritis.

As illustrated in FIGS. 4A to 4D, it was confirmed that all of the four biomarkers selected in Example 3 were detected at higher levels in the serum samples of all types of patients with rheumatoid arthritis than in the serum samples of the control group.

### Example 4-3: Logistic Regression Analysis

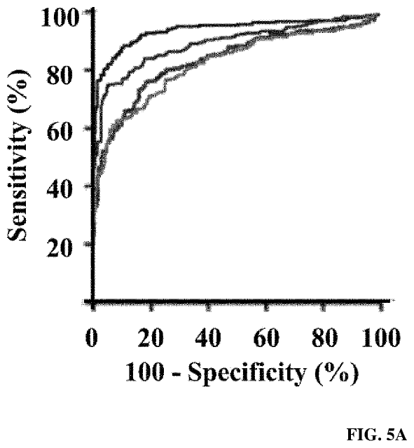

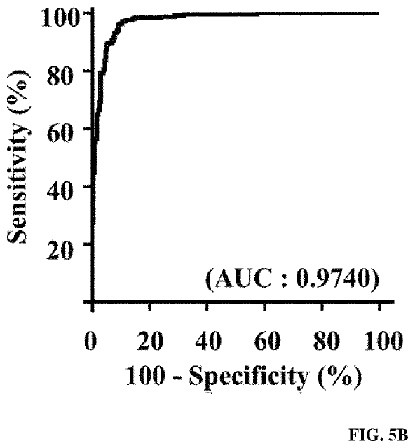

The results acquired by individually measuring the expression levels of the four biomarkers (angiotensinogen, serum amyloid A-4, retinol-binding protein 4, and vitamin D-binding protein) selected in Example 3 in the serum samples of the control group and the experimental group collected in Example 1 were collected, and logistic regression analysis was performed on each of these to measure the diagnostic success rate among all patients (Tables 1 to 4 and FIG. 5A).

TABLE 1
Results of diagnosis using angiotensinogen by regression analysis

| | Diagnosed | Diagnosed | Diagnostic | |
|---|---|---|---|---|
| | as healthy | as patient | success rate | AUC |
| Control | 209 | 42 | 83.3% | 0.8346 |
| group | | | | |
| Experimental | 61 | 169 | 73.5% | |
| group | | | | |

TABLE 2
Results of diagnosis using serum amyloid
A-4 by regression analysis

| | Diagnosed | Diagnosed | Diagnostic | |
|---|---|---|---|---|
| | as healthy | as patient | success rate | AUC |
| Control | 223 | 28 | 88.8% | 0.8890 |
| group | | | | |
| Experimental | 54 | 176 | 76.5% | |
| group | | | | |

TABLE 3
Results of diagnosis using retinol-binding
protein 4 by regression analysis

| | Diagnosed | Diagnosed | Diagnostic | |
|---|---|---|---|---|
| | as healthy | as patient | success rate | AUC |
| Control | 204 | 47 | 81.3% | 0.8170 |
| group | | | | |
| Experimental | 76 | 154 | 67.0% | |
| group | | | | |

TABLE 4
Results of diagnosis using vitamin D-
binding protein by regression analysis

| | Diagnosed | Diagnosed | Diagnostic | |
|---|---|---|---|---|
| | as healthy | as patient | success rate | AUC |
| Control | 228 | 23 | 90.8% | 0.9430 |
| group | | | | |
| Experimental | 32 | 198 | 86.0% | |
| group | | | | |

FIG. 5A is an ROC graph illustrating the results of logistic regression analysis performed on the expression levels of four biomarkers (angiotensinogen, serum amyloid A-4, retinol-binding protein 4, and vitamin D-binding protein) in the serum samples of the control group and the experimental group.

As presented in Tables 1 to 4 and FIG. 5A, when four biomarkers were used, it was confirmed that the diagnostic success rate in healthy controls was 81.3% to 90.8%, the diagnostic success rate in patients with rheumatoid arthritis was 67.0% to 86.0%, and the AUC was 0.8170 to 0.9430.

In order to examine whether there is a change in the diagnostic success rate when the expression levels of the four biomarkers are combined, the results acquired by individually measuring the expression levels of the four biomarkers (angiotensinogen, serum amyloid A-4, retinol-binding protein 4, and vitamin D-binding protein) selected in Example 3 in the serum samples of the control group and the experimental group collected in Example 1 were collected, and logistic regression analysis was performed on the combination of these results to measure the diagnostic success rate among all patients (Table 5 and FIG. 5B).

TABLE 5
Results of diagnosis using four biomarkers by regression analysis

| | Diagnosed | Diagnosed | Diagnostic | |
|---|---|---|---|---|
| | as healthy | as patient | success rate | AUC |
| Control | 234 | 17 | 93.2% | 0.9740 |
| group | | | | |
| Experimental | 19 | 211 | 91.7% | |
| group | | | | |

FIG. 5B is an ROC graph illustrating the results of logistic regression analysis performed on the combination of the expression levels of four biomarkers (angiotensinogen, serum amyloid A-4, retinol-binding protein 4, and vitamin D-binding protein) in the serum samples of the control group and the experimental group,

As presented in Table 5 and FIG. 5B, when four biomarkers were used in combination, it was confirmed that the diagnostic success rate in healthy controls was 93.2%, the diagnostic success rate in patients with rheumatoid arthritis was 91.7%, and the AUC was 0.9740.

Summarizing the results, it has been analyzed that the diagnostic success rate of rheumatoid arthritis can be significantly improved in the case of using four biomarkers provided by the present invention in combination compared to in the case of using the four biomarkers individually.

Based on the above description, it will be understood by those skilled in the art that the present disclosure may be implemented in a different specific form without changing the technical spirit or essential characteristics thereof. Therefore, it should be understood that the above embodiment is not limitative, but illustrative in all aspects. The scope of the disclosure is defined by the appended claims rather than by the description preceding them, and therefore all changes and modifications that fall within metes and bounds of the claims or equivalents of such metes and bounds are therefore intended to be embraced by the claims.

1. A method for providing information necessary to determine occurrence of rheumatoid arthritis, the method comprising:
(a) quantitatively analyzing an expression level of angiotensinogen (AGT) in a serum sample of an individual suspected of having rheumatoid arthritis; and
(b) correlating the quantitatively analyzed expression level of angiotensinogen with determination of occurrence of rheumatoid arthritis.

2. The method according to claim 1, wherein the correlating step (b) is performed by applying a quantitatively analyzed expression level of angiotensinogen to an analysis method selected from the group consisting of a logistic regression method; a linear or nonlinear regression analysis method; a linear or nonlinear classification analysis method; ANOVA; a neural network analysis method; a genetic analysis method; a support vector machine, analysis method; a hierarchical duster analysis or cluster analysis method; a hierarchical algorithm using decision trees, or Kernel principal component analysis method; a Markov Blanket analysis method; a recursive feature elimination or entropy-based recursive feature elimination analysis method; a forward floating search or backward floating search analysis method; and a combination thereof,

3. The method according to claim 1, wherein the step (a) further includes quantitatively analyzing an expression level of a protein selected from the group consisting of retinol-binding protein 4 (RBP4), serum amyloid A-4 (SAA4), vitamin D-binding protein (VDBP), and a combination thereof.

4. The method according to any one of claims 1 to 3, wherein the step (b) further includes correlating an expression level of a protein selected from the group consisting of retinol-binding protein 4, serum amyloid A-4, vitamin D-binding protein, and a combination thereof with determination of occurrence of rheumatoid arthritis.

5. The method according to claim 4, wherein the correlating step is performed by combining quantitative analysis results of angiotensinogen, retinol-binding protein 4, serum amyloid A-4, and vitamin D-binding protein.

6. The method according to claim 5, wherein the combination of quantitative analysis results is performed using a computer algorithm.
